(19) United States
(12) Patent Application Publication
(10) Pub. No.: US 2017/0181636 A1
(43) Pub. Date: Jun. 29, 2017

(54) SYSTEMS FOR IMAGING OF BLOOD FLOW IN LAPAROSCOPY

(71) Applicant: COVIDIEN LP, MANSFIELD, MA (US)

(72) Inventors: ZHONGCHI LUO, SHANGHAI (CN); XINMIN WU, SHANGHAI (CN)

(21) Appl. No.: 15/313,616

(22) PCT Filed: May 23, 2014
(86) PCT No.: PCT/CN2014/078222

§ 371 Date: Nov. 23, 2016

(51) Int. Cl. A61B 5/00; A61B 5/026

(52) U.S. Cl. CPC A61B 5/0084; A61B 5/0261; A61B 5/0059; A61B 5/6847

(57) ABSTRACT

A laparoscopic apparatus for imaging subsurface blood flow of tissue, the laparoscopic apparatus including a light source emitting white light via a light guide, a laser source emitting laser light via an optical fiber and a laparoscope that alternatively receives reflected laser light emitted from the laser source, and reflected white light emitted from the light source. The apparatus further includes a computing device receives the sensed resulted from the laparoscope and generates laser speckle contrast images or white light images according to an output status of the light source and the laser source, and a display that is operatively associated with the computing device and that displays at least one of the laser speckle contrast images and the white light images, where the laser speckle contrast images show the subsurface blood flow.

### CROSS REFERENCE TO RELATED APPLICATION

The present application is a U.S. National Stage application under 35 U.S.C. §371(a) of PCT/CN2014/078222 filed May 23, 2014, the entire contents of which are incorporated by reference herein.

### BACKGROUND

Technical Field

The present disclosure relates to systems for imaging blood flow. More particularly, the present disclosure relates to systems that image subsurface blood flows of tissue with a large field of view.

Discussion of Related Art

Recently, surgeries to internal organs have been greatly increased due to improvements on medical diagnosis, clinicians' operational techniques, medical instruments, and medical systems. Since sufficient blood flow is of fundamental importance in promoting healing after surgery and avoiding leakage of the organ, visually finding and confirming a proper location for surgery is also important. Compared to open surgeries, laparoscopic surgeries leave a relatively small suture on the skin, which results in less pain while recovering and shorter recovery time than open surgeries. For these reasons, more and more patients and clinicians prefer laparoscopic surgeries to open surgeries.

During laparoscopic surgeries, proper anastomosis locations are an important factor for a faster and less damaging recovery after laparoscopic surgeries. However, clinicians have a very limited field of view with a laparoscope due to the limited range of movement and viewing area provided by the laparoscope. For this reason, clinicians have to spend times to find a proper anastomosis location for a laparoscopic surgery. However, even if the clinicians determines a proper anastomosis location, clinicians' visual determination of the proper anastomosis location which receives a sufficient blood flow may not be accurate because clinicians' eyes can see the size of blood vessel but cannot see blood flows inside of the blood vessel. Current practices for assessing local blood supply either indicate the static presence of blood content using narrowband imaging technique or blood flow at a single spot when a fiber optic laser Doppler flowmeter or a fluorescent dye approved for venous injection is used. However, none of these options provide a quantitative mapping of local blood flow dynamics so that clinicians can quickly and accurately assess the blood supply to the tissue under treatment.

### SUMMARY

The present disclosure is directed to laparoscope using laser speckle contrast image techniques which allows a clinician to see a quantitative mapping of local blood flow dynamics in a wide area so that the clinician can quickly and accurately assess the blood supply to a portion under treatment. In an embodiment, the present disclosure is directed to a laparoscopic apparatus for imaging subsurface blood flow of tissue, the laparoscopic apparatus including a light source emitting white light via a light guide, a laser source emitting laser light via an optical fiber, and a laparoscope that alternatively receives reflected laser light emitted from the laser source, and reflected white light emitted from the light source. The laparoscopic apparatus further includes a computing device receives the sensed resulted from the laparoscope and generates laser speckle contrast images or white light images according to an output status of the light source and the laser source, and a display that is operatively associated with the computing device and that displays at least one of the laser speckle contrast images and the white light images, wherein the laser speckle contrast images show the subsurface blood flow. The laparoscopic apparatus may further include a laparoscopic shaft housing the light source and the light guide, and a laparoscopic probe housing the laser source and the optical fiber.

In a further embodiment, the laparoscopic apparatus includes a light sensor that is positioned at a distal end of the first laparoscopic shaft and that senses the reflected laser light and the reflected white light, wherein a wavelength of the laser light is within a sensitivity range of the light sensor. Further, the laparoscopic probe with the optical fiber may be separate from the laparoscopic shaft with white light guide.

In accordance with a further embodiment of the present disclosure, a lens is mounted at the distal end of the optical fiber to expand the laser light to form a laser beam having a field of view. The field of view of the laser light source may be similar to a field of view of the light source. According to some embodiments, the lens is a mirror with a curved surface to deflect the laser beam to a target area. Alternatively, the lens is a prism to deflect the laser beam to a target area.

In a further embodiment the laparoscopic apparatus includes a switch that turns on or off the laser source, and a shutter that is operatively connected to the switch and that opens an aperture and closes the aperture to prevent transmission of the white light to the light guide. The shutter opens the aperture when the switch turns off and the shutter closes the aperture when the switch turns on. The display displays the laser speckle contrast images or white light images of the tissue based on a switching status of the switch and the shutter. Specifically, the display displays the laser speckle contrast images when the switching status of the switch indicates that the switch is on and displays the white light images when the switching status of the switch indicates that the shutter is on.

The wavelength of the laser light may be outside the visible spectrum. For example, the laser may be in a near infrared range. And according to at least one embodiment the optical fiber and the light guide are integrated into a single laparoscopic shaft. Further,

In yet another embodiment, the laser speckle contrast images are based on depth of modulation of speckle intensity fluctuation of a set of pixels. Further the set of pixels may be defined by a time series of intensity of an individual pixel. Still further, the set of pixels may defined by a rectangle window of the display at a time or a cubic window in the (x, y, t) space, wherein x represents the horizontal axis, y represents the vertical axis, and t represents the temporal axis. The subsurface blood flow may be inversely proportional to a squared value of a laser speckle contrast, where the laser speckle contrast is calculated by dividing a variance of the intensity of pixels around the pixel by average intensity of pixels around the pixel. The relationship between the laser speckle and a velocity of the subsurface blood flow may be:

$$K = {[ \frac{1}{2 TV} ( 1 - {e}^{- 2 TV} ) ]}^{\frac{1}{2}} ,$$

where K is the laser speckle, V is a velocity of the subsurface blood flow, and T is an integration time.

Any of the above aspects and embodiments of the present disclosure may be combined without departing from the scope of the present disclosure.

### BRIEF DESCRIPTION OF THE DRAWINGS

Objects and features of the presently disclosed systems and methods will become apparent to those of ordinary skill in the art when descriptions of various embodiments are read with reference to the accompanying drawings, of which:

FIG. 1 is a schematic illustration of a system for imaging subsurface blood flows in accordance with an embodiment of the present disclosure;

FIG. 2A is an image illustrating an image of a portion of the digestive track using white light;

FIG. 2B depicts raw data generated using a laser speckle imaging system in accordance with an embodiment of the present disclosure; and

FIG. 2C is a LSCI image illustrating subsurface blood flows of the same portion of the digestive tract as FIG. 2A incorporating the data of FIG. 2B.

### DETAILED DESCRIPTION

The present disclosure is related to systems for imaging subsurface blood flow of tissue using laser speckle contrast imaging techniques. Although the present disclosure will be described in terms of specific illustrative embodiments, it will be readily apparent to those skilled in this art that various modifications, rearrangements and substitutions may be made without departing from the spirit of the present disclosure. The scope of the present disclosure is defined by the claims appended hereto.

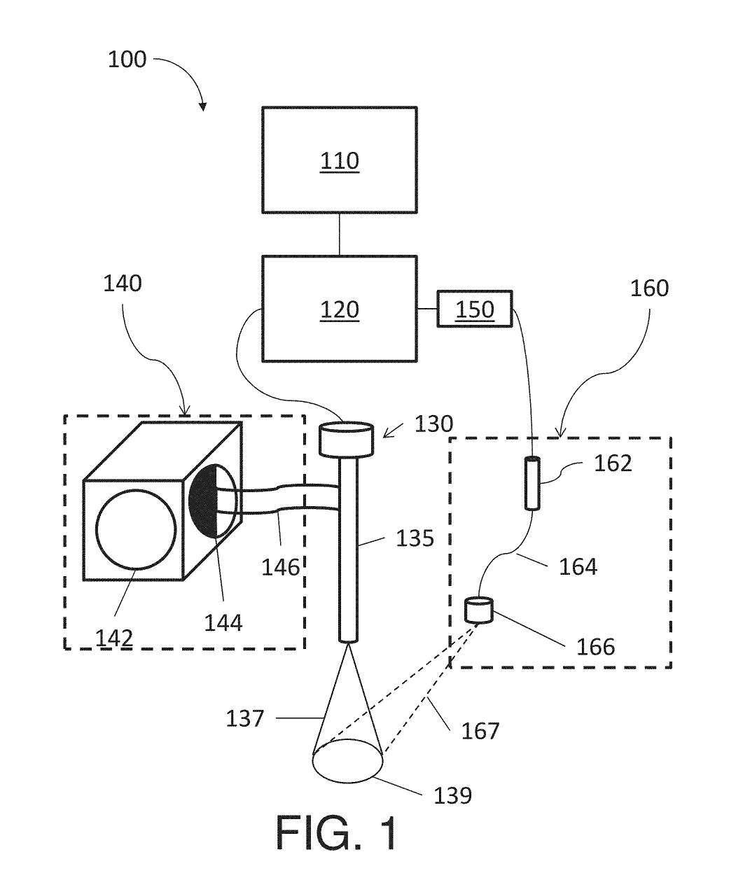

FIG. 1 shows a laparoscopic system 100 that is capable of imaging subsurface blood flow using laser light and imaging inside of a patient's body using white light. The system 100 includes a display 110, a computing device 120, a laparoscope 130, a white light source 140, a switch 150, and a laser light source 160. The display 110 receives video signals from the computing device 120 and displays the video signal. The display 110 may be any form suitable for displaying medical images. The display 110 may be a monitor or a projector.

The computing device 120 is connected to the white light source 150 and to the laser light source 160. When the computing device 120 receives outputs from the laparoscope 130, the computing device 120 converts the outputs into video signals and transmits corresponding video signals to the display 110 so that clinician can see inside of the patient's body. In an aspect, the computing device 120 performs image enhancing processes such as noise reduction, pseudo color rendering, etc.

Whether the white light or laser light is used, the laparoscope 130 receives and projects light reflected from the tissue onto its image sensor such as CCD or CMOS array and outputs the sensed result to the computing device 120. The computing device 120 processes the received light (either white or laser) and generates either white light images using standard image processing techniques or laser speckle contrast images using the laser speckle contrast imaging techniques. The laser speckle contrast images depict subsurface blood flow of the internal organs. Thus, clinicians can see which part of the internal organs have sufficient blood supply so that the clinicians may be able to identify a proper anastomosis location on an internal organ (e.g., a digestive track) by looking at the LSCI images real-time.

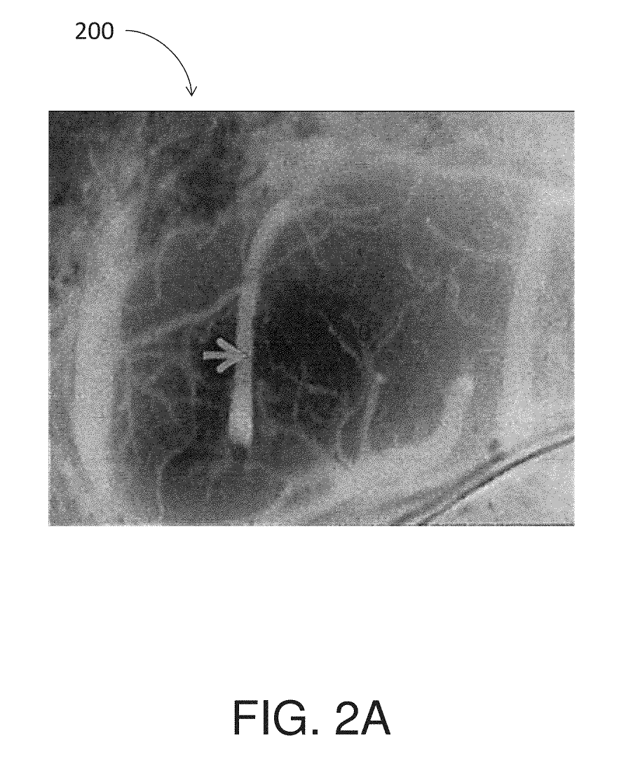

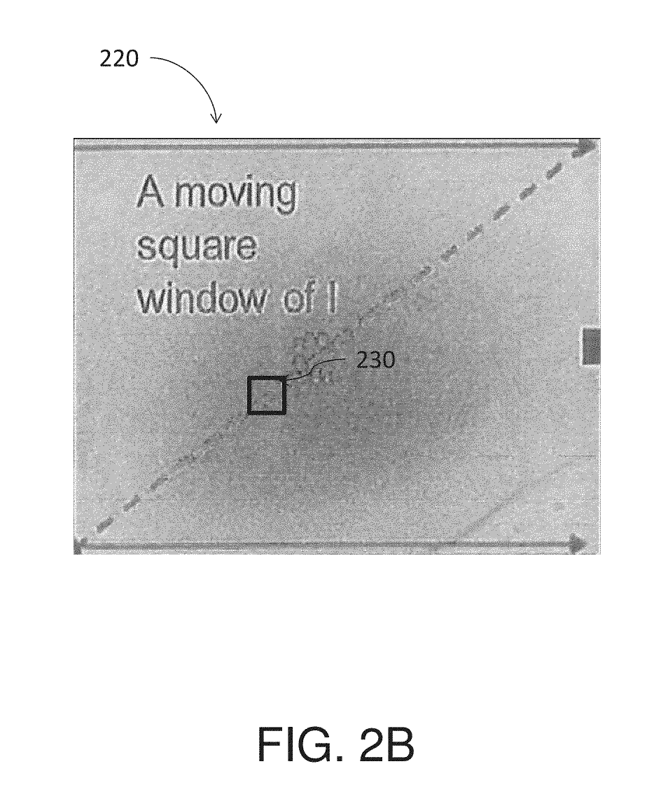

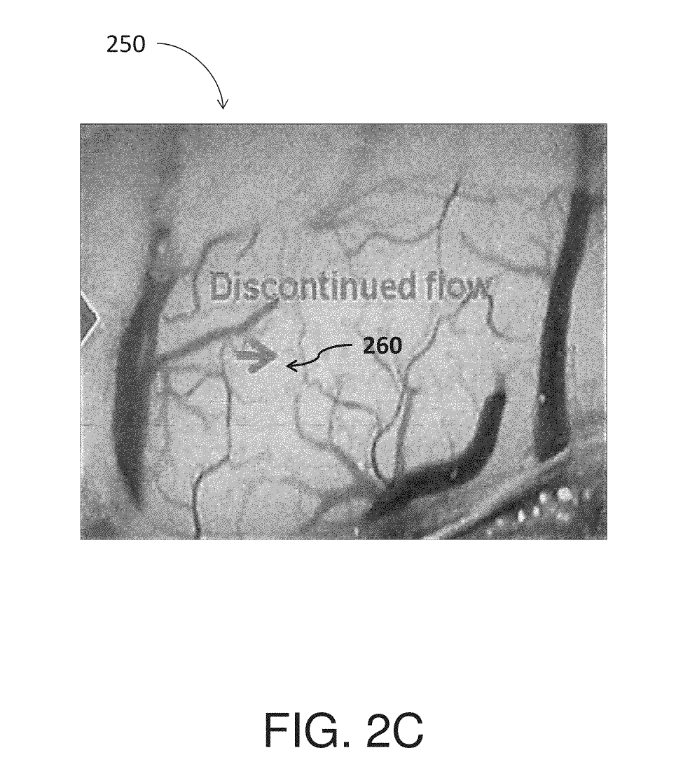

FIG. 2A depicts an image 200 illustrating a wide area of internal organs using white light from the laparoscopic system 100 of FIG. 1. A blood vessel 210 in the left side of the center of the image 200 is identified by an arrow. Visually, the identified blood vessel 210 appears relatively large compared to small branches of surrounding vessels. A clinician might erroneously judge by looking at this visual image that the identified blood vessel 210 is supplying much more blood than the other small branches of surrounding vessels. However, the size of blood vessel is not determinative of the amount of blood it supplies to the organ. The limitations on blood flow may be caused by any of a variety of bio-physiological reasons, such as occluded blood vessel, or other vessel-related diseases. As can be appreciated, accurate, non-invasive determinations of blood flow of that blood vessel is highly desirable.

FIG. 2B depicts a representation of raw laser speckle data 220 of the internal organ, which is shown in FIG. 2A, using laser light. Laser light has lights having same frequency but different phases and amplitudes, which add together to give a pattern in which its amplitude and intensity vary randomly. Thus, the laser speckle pattern generally has a Gaussian distribution pattern of intensity. The laser speckle data is not readily readable by a clinician and it would be exceedingly difficult to analyze such data in real time and identify objects in the images. For example, the same location of the blood vessel 210 of FIG. 2A is identified by a box referenced by the arrow 230 in FIG. 2B. However, it is very difficult to identify the location of blood vessel in FIG. 2B such that the clinician can use the data displayed.

Nevertheless, when there is a moving object in the area illuminated by the laser light source, the intensity fluctuates according to the movement of the moving object (e.g., circulating red blood cells) and thus forms a pattern different from the Gaussian distribution pattern. The laser speckle contrast imaging techniques uses these speckle patterns that are resulted from the moving objects or the interference of many waves having the same frequency. By analyzing the intensity fluctuation of these laser speckle patterns together with time, velocity of the moving object can be identified. More detailed description of this technique for identifying velocities in a wide area is described in “Laser Doppler, speckle and related techniques for blood perfusion mapping and imaging” by J. David Briers. As a result, the data of FIG. 2B can be converted to the image of FIG. 2C utilizing the following techniques to render an image that is useful to a clinician in practice.

The statistics of noise-like raw laser speckle data 220 is related to speckle contrast K containing time component. Specifically, the speckle contrast K includes three variables x, y, and t, where x, y, and t represents horizontal, vertical, and temporal position in the sampling space of the laser light. The speckle contrast K(x, y, t) may be defined by a ratio of the standard deviation σ to the mean intensity I as follows:

$$K ( x , y , t ) = \frac{σ ( x , y , t )}{AVG ( I ( x , y , t ) )} ,$$

where σ(x, y, t) is the standard deviation of intensity in spatial and time domain, I(x, y, t) is the intensity values of a set of pixels adjacent to position (x, y, t) in spatial and time domain, and AVG(I(x, y, t)) is the mean or average intensity of the set of pixels adjacent to the position (x, y, t). In embodiments, the set of pixels may be defined by a time series of intensity of an individual pixel, pixels in a rectangular window in the (x, y) plane at time t, or a consecutive cubic in the (x, y, t) space.

The depth of modulation of the speckle intensity fluctuations generally gives some indication of how much of the laser light is being scattered from moving objects and how much from stationary objects. Further, the frequency spectrum of the fluctuations depends on velocity distribution of the movements of the moving objects. It follows that the speckle contrast K is related to velocity of moving objects or simply subsurface blood flows here. The speckle contrast K is then expressed as follows according to equation:

$$K = {[ \frac{{τ}_{c}}{2 T} ( 1 - {e}^{( \frac{- 2 T}{{τ}_{c}} )} ) ]}^{\frac{1}{2}} ,$$

where T is an integration time and τc is a correlation time. Velocity V is a reciprocal of the correlation time τ_{c}. Thus, the speckle contrast K becomes:

$$K = {[ \frac{1}{2 TV} ( 1 - {e}^{- 2 TV} ) ]}^{\frac{1}{2}} .$$

According to this equation, when the velocity V increases, the exponential term e^{-2TV} is going to be closer to zero and the speckle contrast K is going to increase to a value which is less than

$$\sqrt{\frac{1}{2 TV}} .$$

Since the velocity V is assumed to be greater than or equal to zero, the speckle contrast K is greater than or equal to zero, and bound by

$$\sqrt{\frac{1}{2 TV}} .$$

Based on the equation of the speckle contrast K and the velocity, the squared value of the speckle contrast K is inversely proportional to the velocity V, when assuming that the exponential term e^{-2TV} is comparatively small. Or, in other words, the value

$$\frac{1}{{K}^{2}}$$

is linearly proportional to the velocity V.

The computing device 120 of the laparoscopic system 100 normalizes the value

$$\frac{1}{{K}^{2} ( x , y , t )}$$

and converts the normalized value into intensity of a pixel (x, y) of the laser speckle contrast image. Since

$$\frac{1}{{K}^{2} ( x , y , t )}$$

is inversely proportional to the velocity V, if

$$\frac{1}{{K}^{2} ( x , y , t )}$$

is small, the velocity is also small and intensity of the pixel (x, y) is low and, if

$$\frac{1}{{K}^{2} ( x , y , t )}$$

is large, the velocity V is correspondingly large and the intensity of the pixel (x, y) is high. Thus, a portion of vessel where the blood flows slowly is illustrated darker than a portion of vessel where the blood flows fast. However, the way of converting laser speckle into intensity is not limited by the equation presented above but is provided as an example. Any correlation between the laser speckle and intensity can be made within the scope of this disclosure by a person having ordinary skills in this art.

Further, the intensities of pixels resulted from the laser speckle contrast image processes may be normalized, formatted for display, stored, and passed to other processes such as noise reduction, pseudo color rendering, or fusion with white light image, etc.

FIG. 2C is a laser speckle contrast image 250 produced from the raw laser speckle image data 220 of FIG. 2B, which is readily usable by a clinician to quantify blood flows in the whole view of the image. Contrary to the visual interpretation of the image 200 of FIG. 2A, the blood vessel 260, which corresponds to the blood vessel 210 identified by the arrow of the image 200, is illustrated dark, meaning that the blood vessel 210 identified by the arrow has very low blood flow or no blood flow at all. On the other hand, the laser speckle contrast image illustrates that the lower right and the right vessels, which are bigger than the other vessels surrounding these two vessels, have very high intensity, meaning that these vessels have very high blood flows. Based on this laser speckle contrast image 250, clinicians may easily decide the desirability of performing anastomosis in that portion of the organ based on the detailed information provided by the laser speckle contrast image. In this way, the laser speckle contrast image provides further information of blood flows in a wide area by varying intensity based on velocity of subsurface blood flow dynamics so that clinicians can easily identify a proper portion for an operation.

These techniques for processing the laser speckle can be implemented in the computing device 120 which may be a personal computer, tablet, server, or computing equipment having a processor that performs image-related processes. The computing device 120 may include at least one processor and at least one memory (e.g., hard drive, read-only memory, random access memory, mountable memory, etc.) that stores data and programs. When the programs are executed, the programs are loaded into a memory in a form of computer-executable instructions. At least one processor may execute the computer-executable instructions to perform functionalities of the programs. Functionalities of one program may include visual image processes and functionalities of another program may include the LSCI processes. Or one program may have both functionalities of visual image processes and LSCI processes based on which is input to the computing device 120 from the laparoscope 130 and the laser light provider 160.

The laparoscope 130 may be connected to the computing device 120 wired or wirelessly. The laparoscope 130 includes a laparoscopic shaft 135 which can be inserted into the inside of a patient's body via an incision or an opening of the patient's body. Inside of the laparoscopic shaft 135 may be hollow so that medical instruments other than a light guide, such as ablating antenna, scissors, gas tube for insufflating the inside of the patient's body, suctioning device, hooks, and/or tissue sealing device, can be inserted through the laparoscopic shaft 135 into the inside of the patient's body.

When white light is guided by the light guide of the laparoscopic shaft 135 to the distal end of the light guide, the light disperses to illuminate an area of interest 139 based on the topology of the distal end of the light guide or based on materials covering the distal end of the light guide. A clinician may rotate or move the laparoscopic shaft 135 to visually see the area of interest 139. The light may be directed to another area of interest by moving the laparoscopic shaft 135. The distal end of the light guide may be flat, curved, recessed, or convex. The distal end of the light guide may be connected to or covered by a lens to form a directed light beam.

A light sensor (which is not shown) may be also located at the distal end of the laparoscopic shaft 135, which senses lights reflected, scattered, or absorbed from the area of interest 139. The light sensor then provides the sensed results to the computing device 120 via the wired or wireless connection. The light sensor may be a color charged device (CCD), photovoltaic device, CMOS, or light detector. The light sensor can sense a specific range of wavelengths. For example, a range of wavelength sensitivity of a CCD may be wider than the range of the visible white light (e.g., 400 nm to 700 nm). The light sensor may include a lens that receives light reflected, scatter, and/or absorbed from internal organs and the light sensors receive the light via the lens. The lens of the light sensor may cover the field of view 137 so that sufficient area of interest can be sensed. Further the lens of the light sensor forms a field of view 137 which defines a size and resolution of image that the computing device 120 can generate.

The laparoscope 130 may further include a wired or wireless transmitter that transmits the sensed results to the computing device 120.

The white light source 140 provides white light to the laparoscope 130 and includes a white light generator 142, a shutter 144, and a light guide 146. The white light generator 142 generates white light having a wavelength of about 400 nm to about 700 nm, (i.e., visible to human eyes). The generated white light may help a clinician to see internal organs of the patient's body illuminated in the area of interest 139 so that the clinician can make an appropriate diagnosis and perform proper medical operations at an intended location. The white light generator 142 may be fluorescent lamps, compact fluorescent lamps (CFL), cold cathode fluorescent lamps (CCFL), high-intensity discharge lamps, light-emitting diode (LED) lamps, or incandescent lamps. The white light generator 142, however, is not limited by this list but may be any light source that can generate white light.

The shutter 144 opens and closes an aperture to control transmission of the white light to the light guide 146. For example, when the shutter 144 closes the aperture, the white light generated by the white light source 142 is not transmitted to the laparoscope 130 via the light guide 146 and, when the shutter 144 opens the aperture, the generated white light is transmitted to the laparoscope 130. In this way, the laparoscope 130 may be selectively used to see internal organs with the white light.

In embodiments, the white light source 140 may be any light source that can generate light that can be sensed by a sensor and the sensed results can be processed by the computing device 120 to generate images of the internal organs suitable for any medical treatments.

The switch 150 is connected to the computing device 120 and the laser light source 160 controls transmitting outputs of the laser light source 160, and transmit the switch status to the computing device 120. When the switch 150 transmits an on position as its switch status, the laser light source 160 is turned on and transmits laser light which is reflected from internal organs of the patient and sensed via the laparoscope 130 to the computing device 120 to produce laser speckle images. When the switch 150 transmits an off position as its switch status, the laser light source 160 is turned off and does not transmit laser light.

The laser light source 160 includes a laser light generator 162, an optical fiber 164, and a lens 166. The laser light generator 162 is connected to the switch 150 so that the laser light generator 162 is turned on when the switch 150 is on and vice versa. The laser light generator 162 may be a single mode laser source. The wavelength of the laser light generated by the laser light generator 162 may overlap with the wavelengths of the white light generated by the white light generator 142. In one aspect, the wavelength of the laser light may be in the near infrared range. In another aspect, the wavelength of the laser light may be in any sensitivity range that the laparoscope 130 can handle.

The optical fiber 164 connects the laser light generator 162 and the lens 166. The optical fiber 164 is a flexible, transparent fiber made of high quality extruded glass or plastic, and functions as a waveguide or light pipe to transmit the laser light between the laser light generator 162 and the lens 166. In an aspect, the laparoscope 130 and the laser light source 160 (which may be embodied on a second laparoscopic probe) are inserted separately into the patient through two incisions or two openings one for insertion of the laparoscopic shaft 135 and one for the second laparoscopic probe of the optical fiber 164.

In an aspect, the optical fiber 164 may be integrated into the laparoscopic shaft 135 so that only one opening or incision of the patient is used to image internal organs using both the white light and the laser light. The optical fiber 164 may be also inserted into the laparoscopic shaft 135 via a working channel. In another aspect, the optical fiber 164 may be attached to the outside of the laparoscopic shaft 135. In such a scenario, only one incision would be required in the patient to perform both types of visualization.

The lens 166 is located at the distal end of the optical fiber 164 and expands the laser light generated by the laser light source 162 into a laser beam having a field of view 167. The field of view 167 may be identical, slightly less than, or slightly larger than the field of view 137 of the white light source 140. The lens 166 may be a mirror with a curved surface to deflect the laser beam to a target area of the internal organs which is the area of interest 139. In an aspect, a miniature mirror or prism is attached to between the distal end of the optical fiber 164 and the lens 166 so that the laser light is deflected towards the field of view 167 before being expanded to the laser beam. In another aspect, a miniature mirror or prism having a curved surface may be located between the distal end of the optical fiber 164 and the lens 166 so that the laser light is deflected towards the field of view 367 before being expanded to the laser beam.

In embodiments, the switch 150 may be connected with the white light source 140 and the laser light source 160. The computing device 120 may control the switch 150 in a way that the laser light source 160 is turned on when the shutter 144 closes the aperture, and the laser light source 160 is turned off when the shutter 144 opens the aperture. In other words, the switch 150 may be set to be the on or off position so that the white light source 150 and the laser light source 160 alternatively provides illumination for the laparoscope 230.

In one aspect, the computing device 120 may have a button (not shown) with which a clinician can change a mode of display from a white light mode to a laser light mode or vice versa. Here, pushing the button may trigger the switch 150 to switch form one position to another position to change between the white light source 140 and the laser light source 160, or the switch 150 triggers the button of the computing device 120 to be switched from one position to another. In this way, clinicians can easily change the mode of display as required during laparoscopic surgery by switching back and forth between the white light mode and the laser light mode.

In another aspect, the status of the switch 150 may be interrelated with the computing device 120. When the switch 150 is off, the computing device 120 performs visual imaging processes and, when the switch 150 is on, the computing device 120 performs laser speckle contrast imaging processes. In this way, appropriate imaging processes are performed based on the status of the switch 150.

In embodiments, the laser light source 160 with the switch 150 may be a separate mountable LSCI source, meaning that the mountable LSCI source can be mounted on an existing laparoscopic system to utilize all the features of the existing laparoscopic system and additional features of the mountable LSCI source. In order to utilize features of the mountable LSCI source, a computing device of the existing laparoscopic system may be updated with new software that is capable of performing the LSCI processes to convert raw laser speckle images into laser speckle contrast images.

Further, the wavelength of the laser light generated by the mountable LSCI source should be in the sensitivity range of the existing laparoscopic system. That means the laser light generator 162 may be carefully chosen to meet the sensitivity range of the existing laparoscopic system. In this way, the existing laparoscopic system does not have to replace or change components of the existing laparoscopic system. As far as the wavelength of the laser light is within the sensitivity range of the existing laparoscopic system, any light source may be used for the existing laparoscopic system.

The computing device of the existing laparoscopic system needs to be synchronized with the status of the switch 150 of the mountable LSCI source. That is, when the switch is on, the laser light source is on and the light source of the existing laparoscopic system is off. Also, the computing device correspondingly changes software from standard laparoscopic imaging process to the laser speckle contrast imaging process.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A laparoscopic apparatus for imaging subsurface blood flow of tissue, the laparoscopic apparatus comprising:
a light source emitting white light via a light guide;
a laser source emitting laser light via an optical fiber;
a laparoscope that alternatively receives reflected laser light emitted from the laser source, and receives reflected white light emitted from the light source;
a computing device receives the sensed resulted from the laparoscope and generates laser speckle contrast images or white light images according to an output status of the light source and the laser source; and
a display that is operatively associated with the computing device and that displays at least one of the laser speckle contrast images and the white light images,
wherein the laser speckle contrast images show the subsurface blood flow.

2. The laparoscopic apparatus according to claim 1, further comprising:
a laparoscopic shaft housing the light source and the light guide; and
a laparoscopic probe housing the laser source and the optical fiber.

3. The laparoscopic apparatus according to claim 2, further comprising a light sensor that is positioned at a distal end of the first laparoscopic shaft and that senses the reflected laser light and the reflected white light,
wherein a wavelength of the laser light is within a sensitivity range of the light sensor.

4. The laparoscopic apparatus according to claim 2, wherein the laparoscopic probe with the optical fiber is separate from the laparoscopic shaft with white light guide.

5. The image apparatus according to claim 1, further comprising a lens mounted at the distal end of the optical fiber to expand the laser light to form a laser beam having a field of view.

6. The laparoscopic apparatus according to claim 5, wherein the field of view of the laser light source is similar to a field of view of the light source.

7. The laparoscopic apparatus according to claim 5, wherein the lens is a mirror with a curved surface to deflect the laser beam to a target area.

8. The laparoscopic apparatus according to claim 5, wherein the lens is a prism to deflect the laser beam to a target area.

9. The laparoscopic apparatus according to claim 1, further comprising:
a switch that turns on or off the laser source; and
a shutter that is operatively connected to the switch and that opens an aperture and closes the aperture to prevent transmission of the white light to the light guide,
wherein the shutter opens the aperture when the switch turns off and the shutter closes the aperture when the switch turns on.

10. The laparoscopic apparatus according to claim 9, wherein the display displays the laser speckle contrast images or white light images of the tissue based on a switching status of the switch and the shutter.

11. The laparoscopic apparatus according to claim 10, wherein the display displays the laser speckle contrast images when the switching status of the switch indicates that the switch is on.

12. The laparoscopic apparatus according to claim 10, wherein the display displays the white light images when the switching status of the switch indicates that the shutter is on.

13. The laparoscopic apparatus according to claim 1, wherein a wavelength of the laser light is outside the visible spectrum.

14. The laparoscopic apparatus according to claim 1, wherein a wavelength of the laser light is in a near infrared range.

15. The laparoscopic apparatus according to claim 1, wherein the optical fiber and the light guide are integrated into a single laparoscopic shaft.

16. The laparoscopic apparatus according to claim 1, wherein the laser speckle contrast images are based on depth of modulation of speckle intensity fluctuation of a set of pixels.

17. The laparoscopic apparatus according to claim 16, wherein the set of pixels is defined by a time series of intensity of an individual pixel.

18. The laparoscopic apparatus according to claim 16, wherein the set of pixels is defined by a rectangle window of the display at a time or a cubic window in the (x, y, t) space, wherein x represents the horizontal axis, y represents the vertical axis, and t represents the temporal axis.

19. The laparoscopic apparatus according to claim 18, wherein the subsurface blood flow is inversely proportional to a squared value of a laser speckle contrast,
wherein the laser speckle contrast is calculated by dividing a variance of the intensity of pixels around the pixel by average intensity of pixels around the pixel.

20. The laparoscopic apparatus according to claim 19, wherein a relationship between the laser speckle and a velocity of the subsurface blood flow is:
$$K = {[ \frac{1}{2 TV} ( 1 - {e}^{- 2 TV} ) ]}^{\frac{1}{2}} ,$$
where K is the laser speckle, V is a velocity of the subsurface blood flow, and T is an integration time.
